(19) United States
(12) Patent Application Publication
(10) Pub. No.: US 2009/0221609 A1
(43) Pub. Date: Sep. 3, 2009

(54) Gene Predictors of Response to Metastatic Colorectal Chemotherapy

(75) Inventors: Marguerite Del Rio, Prades Le Lez (FR); Franck Molina, Les Matelles (FR); Bernard Pau, Teyran (FR); Marc Ychou, Montpellier (FR)

Correspondence Address: FOLEY AND LARDNER LLP;SUITE 500, 3000 K STREET NW, WASHINGTON, DC, 20007, US

(73) Assignee: PFIZER PRODUCTS, INC.

(21) Appl. No.: 12/224,335

(22) PCT Filed: Feb. 28, 2007
(86) PCT No.: PCT/US2007/005176

§ 371 Date: Jan. 26, 2009

Related U.S. Application Data
(60) Provisional application No. 60/778,138, filed on Feb. 28, 2006.
(60) Provisional application No. 60/877,516, filed on Dec. 28, 2006.

(51) Int. Cl. A61K 31/519; C12Q 1/68; C40B 30/04; A61K 31/513; A61K 31/4375

(52) U.S. Cl. 514/255.05; 435/6; 506/9; 514/274; 514/283

(57) ABSTRACT

The present invention provides for the identification of genes that are expressed in tumors that are responsive to a given therapeutic regime and whose expression correlates with responsiveness to that therapeutic regime. One or more of the genes of the present invention can be used as markers to identify patients that are likely to be successfully treated by a therapeutic regime.

### BACKGROUND OF THE INVENTION

1. Field of the Invention

The present invention relates generally to the field of cancer biology. More particularly, it concerns gene expression profiles that are indicative of the responsiveness of a patient having cancer to drug therapy.

2. Description of Related Art

Colorectal cancer (CRC) is one of the most common malignant diseases with 945,000 new cases worldwide every year and is the fourth cause of cancer-related deaths worldwide (492,000 deaths/year) (Weitz J, et al., 2005, Lancet 365(9454):153-65). When localized, CRC is often curable by surgery but the prognosis for patients with metastatic disease remains poor. Curative-intent resections can be performed on only 10 to 15% of liver metastases. In the majority of metastatic patients, the standard treatment remains palliative chemotherapy. Fluorouracil-based therapy has been the main treatment for metastatic colorectal cancer for the last 40 years. Major progress has been made by the introduction of regimens containing new cytotoxic drugs, such as irinotecan (Vanhoefer U, et al., 2001, J. Clin. Oncol. 19(5):1501-18) or oxaliplatin (Pelley R J, 2001, Curr. Oncol. Rep. 3(2):147-55). The combinations commonly used, e.g., irinotecan, fluorouracil, and leucovorin (FOLFIRI) and oxaliplatin, fluorouracil, and leucovorin (FOLFOX) can reach an objective response rate of about 50% (Douillard J Y, et al., 2000, Lancet 355 (9209):1041-7; Goldberg R M, et al., 2004, J. Clin. Oncol. 22(1):23-30). However, these new combinations remain inactive in one half of the patients and, in addition, resistance to treatment appear in almost all patients who were initially responders. More recently, two monoclonal antibodies targeting vascular endothelial growth factor Avastin® (bevacizumab) (Genentech Inc., South San Francisco Calif.) an epidermal growth factor receptor Erbitux® (cetuximab) (Imclone Inc. New York City) have been approved for treatment of metastatic colorectal cancer but are always used in combination with standard chemotherapy regimens (Cunningham D, et al., 2004, N. Engl. J. Med. 351(4):337-45; Hurwitz H. et al., 2004, N. Engl. J. Med. 350(23):2335-42).

A major clinical challenge is to identify the subset of patients who will benefit from chemotherapy, both in metastatic and adjuvant settings. The number of anti-cancer drugs and multi-drug combinations has increased substantially in the past decade, however, treatments continue to be applied empirically using a trial-and-error approach. Clinical experience shows that some tumors are sensitive to several different types of chemotherapeutic agents, while other cancers of the same histology show selective sensitivity to certain drugs but resistance to others. There have been many attempts to determine predictive factors of response to drug therapy. Alterations in gene expression, protein expression and polymorphic variants in genes encoding thymidylate synthase, dihydropyrimidine dehydrogenase, and thymidine phosphorylase would be expected to predict a response to fluorouracil (Iacopetta B, et al., 2001, Br. J. Cancer 85(6):827-30; Salonga D, et al., 2000, Clin. Cancer Res. 6(4):1322-7; Kornmann M, et al., 2003, Clin. Cancer Res. 9(11):4116-24). As well, microsatellite-instability status could be an independent predictor of fluorouracil-based adjuvant chemotherapy (Ribic C M, et al., 2003, N. Engl. J. Med. 349(3):247-57). Topoisomerase I expression has been investigated as predictive factor for irinotecan response (Paradiso A, et al., 2004, Int. J. Cancer 111 (2):252-8). High mRNA expression of excision repair cross-complementing rodent repair deficiency, complementation group 1 (includes overlapping antisense sequence) (“ERCC1”) and thymidylate synthase (“TS”) are predictive of poor response to treatment of advanced disease with oxaliplatin plus fluorouracil (Shirota Y, et al., 2001, J. Clin. Oncol. 19(23):4298-304). However, although predictive factor testing is an exciting field of research, it has not yet been routinely applied to clinical practice (Adlard J W, et al., 2002, Lancet Oncol. 3(2):75-82; Ahmed F E., 2005; Expert Rev. Mol. Diagn. 5(3):353-75). Furthermore, an in vitro study on prediction of response of colon cells demonstrated that the measurement of multiple, rather than single marker gene resulted in a more accurate of drug response (Mariadason J M, et al., 2003, Cancer Res. 63(24):8791-812). A test that could assist physicians to select the optimal chemotherapy for a patient from several alternative treatment options would be an important clinical advance.

The application of microarray technology to the cancer field has made possible to obtain large-scale expression profiles in clinical samples. Gene expression profiling has become a strategy to predict clinical outcome or to classify molecular subtype of tumors. Several studies have already been published, showing the feasibility of identifying genes involved in the progression and the prognosis of colorectal cancer (Bertucci F, et al., 2004 Oncogene 23(7):1377-91; Birkenkamp-Demtroder K, et al., 2002, Cancer Res. 62(15):4352-63; Wang Y, et al., 2004, J. Clin. Oncol. 22(9):1564-71; Notterman D A, et al., 2001, Cancer Res. 61(7):3124-30; Eschrich S, et al., 2005, J. Clin. Oncol. 23(15):3526-35) or for predicting drug-response in other cancer types, notably in breast cancer (Chang J C, et al., 2003, Lancet 362(9381):362-9; Iwao-Koizumi K, et al., 2005, J. Clin. Oncol. 23(3):422-31; Jansen M P, et al., 2005, J. Clin. Onco. 23(4):732-40). However, no indication on the possible value of this approach for predicting drug response in colon cancer is presently available (Mariadason J M, et al., 2004, Drug Resist. Updat. 7(3):209-18). Only a recent study showed that gene expression profiling might contribute to the response prediction of rectal adenocarcinomas to preoperative chemoradiotherapy (Ghadimi B M, et al., 2005, J. Clin. Oncol. 23(9):1826-38).

The ability to choose an appropriate treatment at the outset may make the difference between cure and recurrence of a cancer, such as colorectal cancer. The present invention provides for the identification of patients who are the most likely to benefit from drug therapy by assessing the differential expression of one or more of the responsiveness genes in a tumor sample from a patient.

### SUMMARY OF THE INVENTION

The present invention relates generally to the fields of molecular genetics, pharmacogenetics, and cancer therapy. In particular, the present invention is directed to methods for detecting gene expression and correlating the presence or absence of certain genes with responsiveness to chemotherapy. Embodiments of the invention include methods for assessing the responsiveness of a tumor to therapy. In certain embodiments the methods comprise obtaining a sample of a tumor from a patient; evaluating the sample for expression of one or more markers identified in Table 3; and assessing the responsiveness of the tumor to therapy based on the evaluation of marker expression in the sample. Marker herein refers to a gene or gene product (RNA or polypeptide) whose expression is related to response of a cancer to a therapy, either a positive (complete pathological response) or a negative response (residual disease). Expression of a marker may be assessed by detecting polynucleotides or polypeptides derived therefrom. More specifically, the present invention is directed to methods for determining the expression of one or more of the genes listed in Table 3 in a patient with colorectal cancer, and correlating the expression with responsiveness to chemotherapy regimes. The intensity of gene expression detected can be indicative of whether a patient will be a responder or non-responder to a chemotherapy regime. The present invention identifies gene expression profiles associated with colorectal cancer patients who respond to certain pharmaceutical regimes by examining gene expression in tissue from malignant colorectal tissue (primary tumor) of said patients who respond to treatment and those who do not. The present invention also identifies expression profiles which serve as useful diagnostic markers to treatment response and drug efficacy. The present invention also preferably provides a method to assess the responsiveness of a patient with metastatic colorectal cancer to drug therapy.

In certain aspects of the invention, the tumor comprises colorectal cancer. In still other aspects the tumor is sampled by aspiration, biopsy, or surgical resection. Embodiments of the invention include assessing the expression of the one or more markers by detecting a mRNA derived from one or more markers. In a preferred embodiment, detection comprises microarray analysis, and more preferably the microarray is an Affymetrix Gene Chip. In other aspects of the invention, detection comprises nucleic acid amplification, preferably PCR. In still further aspects, detection is by in situ hybridization. In further embodiments, assessing the expression of one or more markers is by detecting a protein derived from a gene identified as a marker. A protein may be detected by immunohistochemistry, western blotting, or other known protein detection means.

A further embodiment includes methods of monitoring a cancer patient receiving chemotherapy. Methods of monitoring a cancer patient comprise obtaining a tumor sample from the patient during chemotherapy; evaluating expression of one or more markers of Table 3 in the tumor sample; and assessing the cancer patient's responsiveness to chemotherapy. A tumor sample may be obtained, evaluated and assessed repeatedly at various time points during chemotherapy (e.g. before, during, and after drug treatment).

Accordingly, in certain aspects it would be useful to identify genes and/or gene products that represent prognostic genes with respect to the response to a given therapeutic agent or class of therapeutic agents. It then may be possible to determine which patients will benefit from a particular therapeutic regimen and, importantly, determine when, if ever, the therapeutic regime begins to lose its effectiveness for a given patient. The ability to make such predictions would make it possible to discontinue a therapeutic regime that has lost its effectiveness well before its loss of effectiveness becomes apparent by conventional measures.

The present invention includes a method of predicting response of a human patient with metastatic colorectal cancer to chemotherapy, comprising detecting the expression of one or more genes selected from the following group LGALS8, SERPINE2, ANGPTL2, ATP50, PRYM, EML2, F8, U2AF1L2, DRD5, GOLGIN-67, ZNF32, PSG9, BOLL, and ZNF583 from a tissue sample from the patient wherein said gene expression is indicative of response to chemotherapy.

Another aspect of the invention includes a method of predicting response of a human patient with metastatic colorectal cancer to chemotherapy, comprising detecting the expression of two or more genes selected from the group LGALS8, SERPINE2, ANGPTL2, ATP50, PRYM, EML2, F8, U2AF1L2, DRD5, GOLGIN-67, ZNF32, PSG9, BOLL, and ZNF583 from a tissue sample from the patient wherein said gene expression is indicative of response to chemotherapy. The term “two or more,” “three or more,” etc means that one can select two or more, or three or more genes from those listed in Table 3 in any order or combination.

In another aspect of the invention a method of predicting response of a human patient with metastatic colorectal cancer to chemotherapy, comprising detecting the expression of three or more genes selected from the group LGALS8, SERPINE2, ANGPTL2, ATP50, PRYM, EML2, F8, U2AF1L2, DRD5, GOLGIN-67, ZNF32, PSG9, BOLL, and ZNF583 from a tissue sample from the patient wherein said gene expression is indicative of response to chemotherapy is included.

A further aspect of the invention includes a method of predicting response of a human patient with metastatic colorectal cancer to chemotherapy, comprising detecting the expression of four or more genes selected from the group LGALS8, SERPINE2, ANGPTL2, ATP50, PRYM, EML2, F8, U2AF1L2, DRD5, GOLGIN-67, ZNF32, PSG9, BOLL, and ZNF583 from a tissue sample from the patient wherein said gene expression is indicative of response to chemotherapy.

This invention also includes a method of predicting response of a human patient with metastatic colorectal cancer to chemotherapy, comprising detecting the expression of five or more genes selected from the group LGALS8, SERPINE2, ANGPTL2, ATP50, PRYM, EML2, F8, U2AF1L2, DRD5, GOLGIN-67, ZNF32, PSG9, BOLL, and ZNF583 from a tissue sample from the patient wherein said gene expression is indicative of response to chemotherapy.

Another aspect of the invention includes a method of predicting response of a human patient with metastatic colorectal cancer to chemotherapy, comprising detecting the expression of six or more genes selected from the group LGALS8, SERPINE2, ANGPTL2, ATP50, PRYM, EML2, F8, U2AF1L2, DRD5, GOLGIN-67, ZNF32, PSG9, BOLL, and ZNF583 from a tissue sample from the patient wherein said gene expression is indicative of response to chemotherapy.

The invention also includes a method of predicting response of a human patient with metastatic colorectal cancer to chemotherapy, comprising detecting the expression of seven or more genes selected from the group LGALS8, SERPINE2, ANGPTL2, ATP50, PRYM, EML2, F8, U2AF1L2, DRD5, GOLGIN-67, ZNF32, PSG9, BOLL, and ZNF583 from a tissue sample from the patient wherein said gene expression is indicative of response to chemotherapy.

Another aspect of the invention includes a method of predicting response of a human patient with metastatic colorectal cancer to chemotherapy, comprising detecting the expression of eight or more genes selected from the group LGALS8, SERPINE2, ANGPTL2, ATP50, PRYM, EML2, F8, U2AF1L2, DRD5, GOLGIN-67, ZNF32, PSG9, BOLL, and ZNF583 from a tissue sample from the patient wherein said gene expression is indicative of response to chemotherapy.

In another aspect, the invention includes a method of predicting response of a human patient with metastatic colorectal cancer to chemotherapy, comprising detecting the expression of nine or more genes selected from the group LGALS8, SERPINE2, ANGPTL2, ATP50, PRYM, EML2, F8, U2AF1L2. DRD5, GOLGIN-67, ZNF32, PSG9, BOLL, and ZNF583 from a tissue sample from the patient wherein said gene expression is indicative of response to chemotherapy.

One embodiment of the invention includes a method of predicting response of a human patient with metastatic colorectal cancer to chemotherapy, comprising detecting the expression of ten or more genes selected from the group LGALS8, SERPINE2, ANGPTL2, ATP50, PRYM, EML2, F8, U2AF1L2, DRD5, GOLGIN-67, ZNF32, PSG9, BOLL, and ZNF583 from a tissue sample from the patient wherein said gene expression is indicative of response to chemotherapy.

In another embodiment of the invention, a method of predicting response of a human patient with metastatic colorectal cancer to chemotherapy, comprising detecting the expression of eleven or more genes selected from the group LGALS8, SERPINE2, ANGPTL2, ATP50, PRYM, EML2, F8, U2AF1L2, DRD5, GOLGIN-67, ZNF32, PSG9, BOLL, and ZNF583 from a tissue sample from the patient wherein said gene expression is indicative of response to chemotherapy in included.

A further embodiment of the invention includes a method of predicting response of a human patient with metastatic colorectal cancer to chemotherapy, comprising detecting the expression of twelve or more genes selected from the group LGALS8, SERPINE2, ANGPTL2, ATP50, PRYM, EML2, F8, U2AF1L2, DRD5, GOLGIN-67, ZNF32, PSG9, BOLL, and ZNF583 from a tissue sample from the patient wherein said gene expression is indicative of response to chemotherapy.

Yet another aspect of the invention includes a method of predicting response of a human patient with metastatic colorectal cancer to chemotherapy, comprising detecting the expression of thirteen or more genes selected from the group LGALS8, SERPINE2, ANGPTL2, ATP50, PRYM, EML2, F8, U2AF1L2, DRD5, GOLGIN-67, ZNF32, PSG9, BOLL, and ZNF583 from a tissue sample from the patient wherein said gene expression is indicative of response to chemotherapy.

Another aspect of the invention includes a method of predicting response of a human patient with metastatic colorectal cancer to chemotherapy, comprising detecting the expression of fourteen or more genes selected from the group LGALS8, SERPINE2, ANGPTL2, ATP50, PRYM, EML2, F8, U2AF1L2, DRD5, GOLGIN-67, ZNF32, PSG9, BOLL, and ZNF583 from a tissue sample from the patient wherein said gene expression is indicative of response to chemotherapy.

This invention also includes a method of predicting response of a human patient with metastatic colorectal cancer to chemotherapy, comprising detecting the expression of a gene selected from the group SERPINE2, ANGPTL2, ATP50, PRYM, EML2, F8, U2AF1L2, DRD5, GOLGIN-67, ZNF32, PSG9, BOLL, and ZNF583 from a tissue sample from the patient wherein said gene expression is indicative of response to chemotherapy.

In another embodiment of the invention, the method can be used to predict response of a human patient with metastatic colorectal cancer to chemotherapy, comprising detecting the expression of a gene selected from the group ANGPTL2, ATP50, PRYM, EML2, F8, U2AF1L2, DRD5, GOLGIN-67, ZNF32, PSG9, BOLL, and ZNF583 from a tissue sample from the patient wherein said gene expression is indicative of response to chemotherapy.

Another embodiment of the invention includes a method of predicting the response of a human patient with metastatic colorectal cancer to chemotherapy, comprising detecting the expression of a gene selected from the group ATP50, PRYM, EML2, F8, U2AF1L2, DRD5, GOLGIN-67, ZNF32, PSG9, BOLL, and ZNF583 from a tissue sample from the patient wherein said gene expression is indicative of response to chemotherapy.

A further embodiment of the invention includes a method of predicting response of a human patient with metastatic colorectal cancer to chemotherapy, comprising detecting the expression of a gene selected from the group PRYM, EML2, F8, U2AF1L2, DRD5, GOLGIN-67, ZNF32, PSG9, BOLL, and ZNF583 from a tissue sample from the patient wherein said gene expression is indicative of response to chemotherapy

Another aspect of the invention includes a method of predicting response of a human patient with metastatic colorectal cancer to chemotherapy, comprising detecting the expression of a gene selected from the group EML2, F8, U2AF1L2, DRD5, GOLGIN-67, ZNF32, PSG9, BOLL, and ZNF583 from a tissue sample from the patient wherein said gene expression is indicative of response to chemotherapy

Yet another embodiment of the invention includes a method of predicting response of a human patient with metastatic colorectal cancer to chemotherapy, comprising detecting the expression of a gene selected from the group F8, U2AF1L2, DRD5, GOLGIN-67, ZNF32, PSG9, BOLL, and ZNF583 from a tissue sample from the patient wherein said gene expression is indicative of response to chemotherapy

Another aspect of the invention includes a method of predicting response of a human patient with metastatic colorectal cancer to chemotherapy, comprising detecting the expression of a gene selected from the group U2AF1L2, DRD5, GOLGIN-67, ZNF32, PSG9, BOLL, and ZNF583 from a tissue sample from the patient wherein said gene expression is indicative of response to chemotherapy

A further aspect of the invention includes a method of predicting response of a human patient with metastatic colorectal cancer to chemotherapy, comprising detecting the expression of a gene selected from the group DRD5, GOLGIN-67, ZNF32, PSG9, BOLL, and ZNF583 from a tissue sample from the patient wherein said gene expression is indicative of response to chemotherapy

Another embodiment of the invention includes a method of predicting response of a human patient with metastatic colorectal cancer to chemotherapy, comprising detecting the expression of a gene selected from the group GOLGIN-67, ZNF32, PSG9, BOLL, and ZNF583 from a tissue sample from the patient wherein said gene expression is indicative of response to chemotherapy

Another embodiment of the invention includes a method of predicting response of a human patient with metastatic colorectal cancer to chemotherapy, comprising detecting the expression of a gene selected from the group ZNF32, PSG9, BOLL, and ZNF583 from a tissue sample from the patient wherein said gene expression is indicative of response to chemotherapy

This invention also includes a method of predicting response of a human patient with metastatic colorectal cancer to chemotherapy, comprising detecting the expression of a gene selected from the group PSG9, BOLL, and ZNF583 from a tissue sample from the patient wherein said gene expression is indicative of response to chemotherapy

Another aspect of the invention includes a method of predicting response of a human patient with metastatic colorectal cancer to chemotherapy, comprising detecting the expression of a gene selected from the group BOLL, and ZNF583 from a tissue sample from the patient wherein said gene expression is indicative of response to chemotherapy

A further aspect of the invention includes methods of predicting response of a human patient with metastatic colorectal cancer to chemotherapy that comprises administering a pharmaceutical regimen of irinotecan, fluorouracil, and leucovorin to the patient. The methods can also be used to predict response of a human patient with metastatic colorectal cancer to chemotherapy that comprises administering a pharmaceutical regimen of oxaliplatin, fluorouracil, and leucovorin to the patient.

This invention also provides for methods of assessing the expression of the one or more of the genes in Table 3 by detecting a protein derived from a gene identified as a marker derived from a sample from said human.

In some aspects, the present invention provides a method of determining a chemotherapy regime for a human patient with metastatic colorectal cancer, comprising detecting the expression of the genes selected from LGALS8, SERPINE2, ANGPTL2, ATP50, PRYM, EML2, F8, U2AF1L2, DRD5, GOLGIN-67, ZNF32, PSG9, BOLL, and ZNF583 from a colon tumor tissue sample from the patient wherein said gene expression is indicative of response to chemotherapy; and administering a pharmaceutical regimen comprising irinotecan, fluorouracil, and leucovorin to said patient if the predictor classifier (previously determined using the SVM-learning algorithm) applied to the expression of the fourteen genes from Table 3 from a tumor tissue sample from the patient classifies the patient as responder patient.

The Support Vector Machines (SVM) are a new type of learning algorithm initiated by Vapnik (1995) and then applied to the microarray data analysis (Ben-Dor et al., 2000, Journal of the Computational Biology, 7, 559-583; Brown et al., 2000, Proc. Natl. Acad. Sci. USA 97:262-267). At first, the aim of the algorithm is to search the best hyperplane that separates the data into two classes. This hyperplane is optimal in the sense that it maximises the distance between the nearest learning points also called support vector. The classification for a new observation is determined by its position with regard to the hyperplane. The nature of statistical learning theory. Springer edition.

When used for classification, the SVM algorithm creates a hyperplane that separates the data into two classes (responders and non responders).

This invention provides a method of determining a chemotherapy regime for a human patient with metastatic colorectal cancer, comprising detecting the expression of the genes selected from LGALS8, SERPINE2, ANGPTL2, ATP50, PRYM, EML2, F8, U2AF1L2, DRD5, GOLGIN-67, ZNF32, PSG9, BOLL, and ZNF583 from a tumor tissue sample from the patient wherein said gene expression is indicative of response to chemotherapy; and administering a pharmaceutical regimen comprising oxaliplatin, fluorouracil, and leucovorin to said patient if the predictor classifier (previously determined using SVM-learning algorithm) applied to the expression of the fourteen genes from Table 3 from a tumoral tissue sample from the patient classifies the patient as non-responder patient.

This invention further provides methods of monitoring response of a human patient with metastatic colorectal cancer to chemotherapy, comprising administering a pharmaceutical regimen to the patient; detecting the expression of one or more of the genes selected from LGALS8, SERPINE2, ANGPTL2, ATP50, PRYM, EML2, F8, U2AF1L2, DRD5, GOLGIN-67, ZNF32, PSG9, BOLL, and ZNF583 from a tumor tissue sample from the patient; and comparing the patient's gene expression detected to the gene expression from a cell population comprising colorectal tumor cells. One such pharmaceutical regime can comprise administering irinotecan, fluorouracil, and leucovorin. Another such pharmaceutical regime can comprise administering oxaliplatin, fluorouracil, and leucovorin.

In another aspect, the present invention provides a method of modifying a chemotherapy treatment for a human patient with metastatic colorectal cancer, comprising administering a pharmaceutical regimen to the patient; detecting the expression of one or more of the genes selected from LGALS8, SERPINE2, ANGPTL2, ATP50, PRYM, EML2, F8, U2AF1L2, DRD5, GOLGIN-67, ZNF32, PSG9, BOLL, and ZNF583 from a colon tumor tissue sample from the patient; and administering FOLFIRI when one or more genes identified are expressed or administering FOLFOX when one or more genes identified are not expressed.

The present invention also contemplates methods for detecting a response of a human patient with metastatic colorectal cancer to chemotherapy.

The invention further comprises kits useful for the practice of one or more of the methods of the invention. In some preferred embodiments, a kit may contain one or more solid supports having attached thereto one or more oligonucleotides. The solid support may be a high-density oligonucleotide array. Kits may further comprise one or more reagents for use with the arrays, one or more signal detection and/or array-processing instruments, one or more gene expression databases and one or more analysis and database management software packages.

The present invention also provides for a kit for use to select the optimal chemotherapy from several alternative treatment options for a human patient with metastatic colorectal cancer, the kit comprising:

a. a microarray for detecting a mRNA derived from a sample from said human to assess the expression of the one or more of the following genes LGALS8, SERPINE2, ANGPTL2, ATP50, PRYM, EML2, F8, U2AF1L2, DRD5, GOLGIN-67, ZNF32, PSG9, BOLL, and ZNF583; and

b. instructions describing a method of using said microarray.

Other embodiments of the invention entail kits wherein the microarray is an Affymetrix® Gene Chip. The invention also contemplates detection by in situ hybridization and detection by nucleic acid amplification.

Another embodiment contemplated by the present invention is a kit for use to select the optimal chemotherapy regime from several alternative treatment options for a human patient with metastatic colorectal cancer, the kit comprising:

a. a microarray for detecting a protein derived from a sample from said human to assess the expression of the one or more of the following genes LGALS8, SERPINE2, ANGPTL2, ATP50, PRYM, EML2, F8, U2AF1L2, DRD5, GOLGIN-67, ZNF32, PSG9, BOLL, and ZNF583; and

b. instructions describing a method of using said microarray.

Other embodiments of the invention include a kit wherein the proteins are detected by western blotting or by immunohistochemistry.

The present invention also provides for a kit for use to select the optimal chemotherapy from several alternative treatment options for a human patient with metastatic colorectal cancer.

It is contemplated that any method or composition described herein can be implemented with respect to any other method or composition described herein.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

FIG. 1: Analysis of gene expression signature by (A) unsupervised clustering and (B) principal Component Analysis

(A): Each column represents a tumor sample and each row represents a gene. Red and green indicate relative high and low expression, respectively;

(B): Principal component analysis (PCA) involves a mathematical procedure that represents the maximum of the data information in reducing the space dimension. This diagram provides 80% of information with only 3 principal components.

FIG. 2: Proportion of misclassification in validation sets as a function of corresponding training-set size

### DETAILED DESCRIPTION OF THE INVENTION

Currently, there are at least four commonly used pre- or post-operative chemotherapy regimens for stage I-III colorectal cancers. Prior to the present invention, there were few tests to select the best regimen for an individual prior to the start of chemotherapy. Typically, treatments were evaluated empirically using a trial-and-error approach. Complete pathologic eradication of colorectal cancer from the colon (and regional lymph nodes) predicts cure with high accuracy. However, this endpoint is only available after completion of the empirically selected chemotherapy. In the case of FOLFIRI chemotherapy, the course of treatment lasts 3 to 6 months, and only between 45-55% of the patients show an objective response (Complete response+Partial response). Douillard J Y, Cunningham D, Roth A D, et al., Lancet 355:1041-1047, 2000; Toumigand C, Andre T, Achille E, et al., J Clin Oncol 22:229-237, 2004.

The ability to choose an appropriate treatment at the outset can make the difference between cure and recurrence of a cancer, such as colorectal cancer (e.g. metastatic colorectal cancer). The present invention provides for the identification of patients who are the most likely to benefit from a therapy, such as FOLFIRI chemotherapy, by assessing the differential expression of one or more of the responsiveness genes in a tumor sample from a patient. In one example, it is estimated that an individual will experience complete pathological response to FOLFIRI therapy with an estimated 100% positive predictive value and 90% negative predictive value. A “predictive value” as used herein is the percentage of patients predicted to have a certain therapeutic outcome that do actually have the predicted therapeutic outcome. A therapeutic outcome may range from cure to no benefit and may include the slowing of tumor growth, a reduction in tumor burden, eradication of the tumor as determined by pathology, and other therapeutic outcomes. This represents a doubling of the chance of achieving complete or partial response (and likely cure) from FOLFIRI chemotherapy from 45-55% in untested patients to 80% in patients who would be selected to receive FOLFIRI chemotherapy on the basis of the inventive methods of the present invention.

The rate of expected objective responses in the population treated with FOLFIRI is 50%. The gene signature obtained by the present invention permits the classification of 100% of the responder (R) and about 92% of the non-responder (NR) patients with a precision of about 80% to 95% as illustrated in Example 5.

For many patients a FOLFIRI regimen represents the best chance of cure over the unselected use of treatments. The predictive test contemplated by the present invention can be used to select patients for this treatment regimen either as pre- or postoperative treatment. These genes alone or in combination can also be used as therapeutic targets to develop novel drugs against colorectal cancer or to modulate and increase the activity of existing therapeutic agents.

The expression level of a set or subset of identified responsiveness gene(s), or the proteins encoded by the responsive genes, can be used to: 1) determine if a tumor can be or is likely to be successfully treated by an agent or combination of agents; 2) determine if a tumor is responding to treatment with an agent or combination of agents; 3) select an appropriate agent or combination of agents for treating a tumor; 4) monitor the effectiveness of an ongoing treatment; and 5) identify new treatments (either single agent or combination of agents). In particular, the identified responsiveness genes can be utilized as markers (surrogate and/or direct) to determine appropriate therapy, to monitor clinical therapy and human trials of a drug being tested for efficacy, and to develop new agents and therapeutic combinations.

In certain embodiments, methods and compositions include genes (markers) that are expressed in cancer cells responsive to a given therapeutic agent and whose expression (either increased expression or decreased expression) correlates with responsiveness to a therapeutic agent, see Table 3. A “responsiveness gene” or “gene marker” as used herein is a gene whose increased expression or decreased expression is correlated with a cell's response to a particular therapy. A response may be either a therapeutic response (sensitivity) or a lack of therapeutic response (residual disease, which may indicate resistance). Accordingly, one or more of the genes of the present invention can be used as markers (or surrogate markers) to identify tumors and tumor cells that are likely to be successfully treated by a therapeutic agent(s). In addition, the markers of the present invention can be used to identify cancers that have become or are at risk of becoming refractory to a treatment. Aspects of the invention include marker sets that can identify patients that are likely to respond or not to respond to a therapy.

In one embodiment, gene expression is assessed by (1) providing a pool of target nucleic acids derived from one or more target genes; (2) hybridizing the nucleic acid sample to an array of probes (including control probes); and (3) detecting nucleic acid hybridization and assessing a relative expression (transcription) level. The present invention provides methods wherein nucleic acid probes are immobilized on a solid support in an organized array. Oligonucleotides can be bound to a support by a variety of processes, including lithography. It is common in the art to refer to such an array as a “chip.”

As used herein, cancer cells, including tumor cells, are “responsive” to a therapeutic agent if its rate of growth is inhibited or the tumor cells die as a result of contact with the therapeutic agent, compared to its growth in the absence of contact with the therapeutic agent. The quality of being responsive to a therapeutic agent is a variable one, with different tumors exhibiting different levels of “responsiveness” to a given therapeutic agent, under different conditions. In one embodiment of the invention, tumors may be predisposed to responsiveness to an agent if one or more of the corresponding responsiveness markers are expressed.

Cancer, including tumor cells, are “non-responsive” to a therapeutic agent if its rate of growth is not inhibited (or inhibited to a very low degree) or cell death is not induced as a result of contact with the therapeutic agent, compared to its growth in the absence of contact with the therapeutic agent. The quality of being non-responsive to a therapeutic agent is a highly variable one, with different tumors exhibiting different levels of “non-responsiveness” to a given therapeutic agent, under different conditions.

As used herein, cancers, including tumor cells, refer to neoplastic or hyperplastic cells.

Cancers include, but are not limited to, mesothelioma, hepatobilliary cancers (hepatic and billiary duct), a primary or secondary CNS tumor, a primary or secondary brain tumor (including pituitary tumors, astrocytomas, meningiomas and medulloblastomas), lung cancer (NSCLC and SCLC), bone cancer, pancreatic cancer, skin cancer, cancer of the head or neck, cutaneous or intraocular melanoma, ovarian cancer, colon cancer, rectal cancer, liver cancer, cancer of the anal region, stomach cancer, gastrointestinal (gastric, colorectal, and duodenal), breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, gastrointestinal stromal tumor (GIST), pancreatic endocrine tumors (such as pheochromocytoma, insulinoma, vasoactive intestinal peptide tumor, islet cell tumor and glucagonoma), carcinoid tumors, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, tumors of the blood vessels (including benign and malignant tumors such as hemangiomas, hemangiosarcomas, hemangioblastomas and lobular capillary hemangiomas) or a combination of one or more of the foregoing cancers.

Many biological functions are accomplished by altering the expression of various genes through transcriptional (e.g., through control of initiation, provision of RNA precursors, RNA processing, etc.) and/or translational control. For example, fundamental biological processes such as cell cycle, cell differentiation and cell death, are often characterized by the variations in the expression levels of groups of genes.

Assay Methods

The present invention provides methods for determining whether a cancer is likely to be sensitive or resistant to a particular therapy or regimen. Although microarray analysis determines the expression levels of thousands of genes in a sample, only a subset of these genes are significantly differentially expressed between cells having different outcomes to therapy. Identifying which of these differentially expressed genes can be used to predict a clinical outcome requires additional analysis.

The genes described in the present invention are genes whose expression varies by a predetermined amount between tumors that are sensitive to a chemotherapy, e.g., FOLFIRI, versus those that are not responsive or less responsive to a chemotherapy. The genes identified may be used in a variety of nucleic acid detection assays to detect or quantitate the expression a gene or multiple genes in a given sample. The following provides detailed descriptions of the genes of interest in the present invention. It is noted that homologs and polymorphic variants of the genes are also contemplated. As described herein, the relative expression of these genes may be measured through nucleic acid hybridization, e.g., microarray analysis. However, other methods of determining expression of the genes are also contemplated. For example, traditional Northern blotting, nuclease protection, RT-PCR and differential display methods can be used for detecting gene expression levels. Those methods are useful for some embodiments of the invention. It is also noted that probes for the following genes can be designed using any appropriate fragment of the full lengths of the nucleic acids sequences of the genes set forth in Table 3.

Gene expression data may be gathered in any way that is available to one of skill in the art. Typically, gene expression data is obtained by employing an array of probes that hybridize to several, and even thousands or more different transcripts. Such arrays are often classified as microarrays or macroarrays depending on the size of each position on the array.

RNA Preparation and Assessment of RNA Quality

One of skill in the art will appreciate that in order to assess the transcription level (and thereby the expression level) of a gene or genes, it is desirable to provide a nucleic acid sample derived from the mRNA transcript(s). As used herein, a nucleic acid derived from a mRNA transcript refers to a nucleic acid for whose synthesis the mRNA transcript or a subsequence thereof has ultimately served as a template. Thus, a cDNA reverse transcribed from an mRNA, an RNA transcribed from the cDNA, a DNA amplified from the cDNA, an RNA transcribed from the amplified DNA, and the like, are all derived from the mRNA transcript. Detection of such derived products is indicative of the presence and abundance of the original transcript in a sample. Thus, suitable samples include, but are not limited to, mRNA transcripts of the gene or genes, cDNA reverse transcribed from the mRNA, cRNA transcribed from the cDNA, and the like.

Where it is desired to quantify the transcription level of one or more genes in a sample, the concentration of the mRNA transcript(s) of the gene or genes is proportional to the transcription level of that gene. Similarly, it is preferred that the hybridization signal intensity be proportional to the amount of hybridized nucleic acid. As described herein, controls can be run to correct for variations introduced in sample preparation and hybridization.

The nucleic acid may be isolated from the sample according to any of a number of methods well known to those of skill in the art. One of skill in the art will appreciate that where expression levels of a gene or genes are to be detected, preferably RNA (mRNA) is isolated. Methods of isolating total mRNA are well known to those of skill in the art. For example, methods of isolation and purification of nucleic acids are described in Sambrook et Al., (1989) Molecular Cloning—A Laboratory Manual, Cold Spring Harbor Laboratory Press which is incorporated herein by reference. Filter based methods for the isolation of mRNA are also known in the art. Examples of commercially available filter-based RNA isolation systems include RNAqueous® (Ambion) and RNeasy (Qiagen). One of skill in the art would appreciate that it is desirable to inhibit or destroy RNase present in homogenates before homogenates can be used.

Frequently, it is desirable to amplify the nucleic acid sample prior to hybridization. One of skill in the art will appreciate that whatever amplification method is used, if a quantitative result is desired, care must be taken to use a method that maintains or controls for the relative frequencies of the amplified nucleic acids.

Methods of “quantitative” amplification are well known to those of skill in the art. For example, quantitative PCR involves simultaneously co-amplifying a known quantity of a control sequence. This provides an internal standard that may be used to calibrate the PCR reaction. The array may then include probes specific to the internal standard for quantification of the amplified nucleic acid.

Other suitable amplification methods include, but are not limited to polymerase chain reaction (PCR) (Innis, et al., 1990), ligase chain reaction (LCR) (see Wu and Wallace, 1989); Landegren, et al., 1988; Barringer, et al., 1990, transcription amplification (Kwoh, et al., 1989), and self-sustained sequence replication (Guatelli, et al., 1990).

In one embodiment, a nucleic acid sample is the total mRNA isolated from a biological sample. The term “biological sample,” as used herein, refers to a sample obtained from an organism or from components (e.g., cells) of an organism, including diseased tissue such as a tumor, a neoplasia or a hyperplasia. The sample may be of any biological tissue or fluid or cells from any organism as well as cells raised in vitro, such as cell lines and tissue culture cells. Frequently the sample will be a “clinical sample,” which is a sample derived from a patient. Such samples include, but are not limited to, blood, blood cells (e.g., white cells), tissue biopsy or fine needle aspiration biopsy samples, urine, peritoneal fluid, and pleural fluid, or cells therefrom. Biological samples may also include sections of tissues such as frozen sections or formalin fixed sections taken for histological purposes.

In a particular embodiment, the sample mRNA is reverse transcribed with a reverse transcriptase, such as SuperScript II (Invitrogen), and a primer consisting of an oligo-dT and a sequence encoding the phage T7 promoter to generate first-strand cDNA. A second-strand DNA is polymerized in the presence of a DNA polymerase, DNA ligase, and RNase H. The resulting double-stranded cDNA may be blunt-ended using T4 DNA polymerase and purified by phenol/chloroform extraction. The double-stranded cDNA is then transcribed into cRNA. Methods for the in vitro transcription of RNA are known in the art and describe in, for example, Van Gelder, et al. (1990) and U.S. Pat. Nos. 5,545,522; 5,716,785; and 5,891,636, all of which are incorporated herein by reference.

If desired, a label may be incorporated into the cRNA when it is transcribed. Those of skill in the art are familiar with methods for labeling nucleic acids. For example, the cRNA may be transcribed in the presence of biotin-ribonucleotides. The BioArray High Yield RNA Transcript Labeling Kit (Enzo Diagnostics) is a commercially available kit for biotinylating cRNA.

It will be appreciated by one of skill in the art that the direct transcription method described above provides an antisense (aRNA) pool. Where antisense RNA is used as the target nucleic acid, the oligonucleotide probes provided in the array are chosen to be complementary to subsequences of the antisense nucleic acids. Conversely, where the target nucleic acid pool is a pool of sense nucleic acids, the oligonucleotide probes are selected to be complementary to subsequences of the sense nucleic acids. Finally, where the nucleic acid pool is double stranded, the probes may be of either sense, as the target nucleic acids include both sense and antisense strands.

To detect hybridization, it is advantageous to employ nucleic acids in combination with an appropriate detection means. Recognition moieties incorporated into primers, incorporated into the amplified product during amplification, or attached to probes are useful in the identification of nucleic acid molecules. A number of different labels may be used for this purpose including, but not limited to, fluorophores, chromophores, radiophores, enzymatic tags, antibodies, chemiluminescence, electroluminescence, and affinity labels. One of skill in the art will recognize that these and other labels can be used with success in this invention.

Examples of affinity labels include, but are not limited to the following: an antibody, an antibody fragment, a receptor protein, a hormone, biotin, Dinitrophenyl (DNP), or any polypeptide/protein molecule that binds to an affinity label.

Examples of enzyme tags include enzymes such as urease, alkaline phosphatase or peroxidase to mention a few. Colorimetric indicator substrates can be employed to provide a detection means visible to the human eye or spectrophotometrically, to identify specific hybridization with complementary nucleic acid-containing samples.

Examples of fluorophores include, but are not limited to, Alexa 350, Alexa 430, AMCA, BODIPY 630/650, BODIPY 650/665, BODIPY-FL, BODIPY-R6G, BODIPY-TMR, BODIPY-TRX, Cascade Blue, Cy2, Cy3, Cy5, 6-FAM, Fluoroscein, HEX, 6-JOE, Oregon Green 488, Oregon Green 500, Oregon Green 514, Pacific Blue, REG, Rhodamine Green, Rhodamine Red, ROX, TAMRA, TET, Tetramethylrhodamine, and Texas Red.

As mentioned above, a label may be incorporated into nucleic acid, e.g., cRNA, when it is transcribed. For example, the cRNA may be transcribed in the presence of biotin-ribonucleotides. The BioArray High Yield RNA Transcript Labeling Kit (Enzo Diagnostics) is a commercially available kit for biotinylating cRNA.

Means of detecting such labels are well known to those of skill in the art. For example, radiolabels may be detected using photographic film or scintillation counters. In other examples, fluorescent markers may be detected using a photodetector to detect emitted light. In still further examples, enzymatic labels are detected by providing the enzyme with a substrate and detecting the reaction product produced by the action of the enzyme on the substrate, and colorimetric labels are detected by simply visualizing the colored label.

So called “direct labels” are detectable labels that are directly attached to or incorporated into the target (sample) nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin-bearing hybrid duplexes providing a label that is easily detected. For a detailed review of methods of labeling nucleic acids and detecting labeled hybridized nucleic acids see Laboratory Techniques in Biochemistry and Molecular Biology (1993).

Hybridization

Nucleic acid hybridization simply involves contacting a probe and target nucleic acid under conditions where the probe and its complementary target can form stable hybrid duplexes through complementary base pairing (see Lockhart et al., 1999, WO 99/32660, for example). The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids.

Under low stringency conditions (e.g., low temperature and/or high salt) hybrid duplexes (e.g., DNA-DNA, RNA-RNA or RNA-DNA) will form even where the annealed sequences are not perfectly complementary.

Thus specificity of hybridization is reduced at lower stringency. Conversely, at higher stringency (e.g., higher temperature or lower salt) successful hybridization requires fewer mismatches. One of skill in the art will appreciate that hybridization conditions may be selected to provide any degree of stringency. Stringency can also be increased by addition of agents such as formamide. Hybridization specificity may be evaluated by comparison of hybridization to the test probes with hybridization to the various controls that can be present (e.g., expression level control, normalization control, mismatch controls, etc.).

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

As used herein, “hybridization,” “hybridizes,” or “capable of hybridizing” is understood to mean the forming of a double or triple stranded molecule or a molecule with partial double or triple stranded nature. The term “anneal” as used herein is synonymous with “hybridize.” The term “hybridization,” “hybridizes,” or “capable of hybridizing” are related to the term “stringent conditions” or “high stringency” and the terms “low stringency” or “low stringency conditions.”

As used herein “stringent conditions” or “high stringency” are those conditions that allow hybridization between or within one or more nucleic acid strands containing complementary sequences, but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as an mRNA or a nucleic acid segment thereof, or detecting at least one specific mRNA transcript or a nucleic acid segment thereof.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acids, the length and nucleobase content of the target sequences, the charge composition of the nucleic acids, and the presence or concentration of formamide, tetramethylammonium chloride or other solvents in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions,” and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

The hybridization conditions selected will depend on the particular circumstances (depending, for example, on the G+C content, type of target nucleic acid, source of nucleic acid, and size of hybridization probe). Optimization of hybridization conditions for the particular application of interest is well known to those of skill in the art. Representative solid phase hybridization methods are disclosed in U.S. Pat. Nos. 5,843,663, 5,900,481, and 5,919,626. Other methods of hybridization that may be used in the practice of the present invention are disclosed in U.S. Pat. Nos. 5,849,481, 5,849,486, and 5,851,772.

Signal Detection

The hybridized nucleic acids are typically detected by detecting one or more labels attached to the sample nucleic acids. The labels may be incorporated by any of a number of means well known to those of skill in the art (for example, see Affymetrix GeneChip® Expression Analysis Technical Manual.)

DNA arrays and gene chip technology provide a means of rapidly screening a large number of nucleic acid samples for their ability to hybridize to a variety of single stranded DNA probes immobilized on a solid substrate. These techniques involve quantitative methods for analyzing large numbers of genes rapidly and accurately. The technology capitalizes on the complementary binding properties of single stranded DNA to screen nucleic acid samples by hybridization (Pease et al., 1994; Fodor et al., 1991). Basically, a DNA array or gene chip consists of a solid substrate upon which an array of single stranded DNA molecules have been attached. For screening, the chip or array is contacted with a single stranded nucleic acid sample (e.g., cRNA), which is allowed to hybridize under stringent conditions. The chip or array is then scanned to determine which probes have hybridized.

The ability to directly synthesize on or attach polynucleotide probes to solid substrates is well known in the art. See U.S. Pat. Nos. 5,837,832 and 5,837,860, both of which are expressly incorporated by reference. A variety of methods have been utilized to either permanently or removably attach the probes to the substrate. Exemplary methods include: the immobilization of biotinylated nucleic acid molecules to avidin/streptavidin coated supports (Holmstrom, 1993), the direct covalent attachment of short, 5′-phosphorylated primers to chemically modified polystyrene plates (Rasmussen et al., 1991), or the precoating of the polystyrene or glass solid phases with poly-L-Lys or poly L-Lys, Phe, followed by the covalent attachment of either amino- or sulfhydryl-modified oligonucleotides using bifunctional crosslinking reagents (Running et al., 1990; Newton et al., 1993). When immobilized onto a substrate, the probes are stabilized and therefore may be used repeatedly.

In general terms, hybridization is performed on an immobilized nucleic acid target or a probe molecule that is attached to a solid surface such as nitrocellulose, nylon membrane or glass. Numerous other matrix materials may be used, including reinforced nitrocellulose membrane, activated quartz, activated glass, polyvinylidene difluoride (PVDF) membrane, polystyrene substrates, polyacrylamide-based substrate, other polymers such as poly(vinyl chloride), poly(methyl methacrylate), poly(dimethyl siloxane), photopolymers (which contain photoreactive species such as nitrenes, carbenes and ketyl radicals capable of forming covalent links with target molecules).

The Affymetrix GeneChip system may be used for hybridization and scanning of the probe arrays. In a preferred embodiment, the Affymetrix U133A array is used in conjunction with Microarray Suite 5.0 for data acquisition and preliminary analysis.

Normalization Controls

Normalization controls are oligonucleotide probes that are complementary to labeled reference oligonucleotides that are added to the nucleic acid sample. The signals obtained from the normalization controls after hybridization provide a control for variations in hybridization conditions, label intensity, “reading” efficiency and other factors that may cause the hybridization signal to vary between arrays. For example, signals read from all other probes in the array can be divided by the signal from the control probes thereby normalizing the measurements.

Virtually any probe may serve as a normalization control. However, it is recognized that hybridization efficiency varies with base composition and probe length. Preferred normalization probes are selected to reflect the average length of the other probes present in the array, however, they can be selected to cover a range of lengths. The normalization control(s) can also be selected to reflect the (average) base composition of the other probes in the array, however in a preferred embodiment, only one or a few normalization probes are used and they are selected such that they hybridize well (i.e. no secondary structure) and do not match any target-specific probes. Normalization probes can be localized at any position in the array or at multiple positions throughout the array to control for spatial variation in hybridization efficiently.

In a particular embodiment, a standard probe cocktail supplied by Affymetrix is added to the hybridization to control for hybridization efficiency when using Affymetrix Gene Chip arrays.

Expression Level Controls

Expression level controls are probes that hybridize specifically with constitutively expressed genes in the sample. The expression level controls can be used to evaluate the efficiency of cRNA preparation.

Virtually any constitutively expressed gene provides a suitable target for expression level controls. Typically expression level control probes have sequences complementary to subsequences of constitutively expressed “housekeeping genes.”

In one embodiment, the ratio of the signal obtained for a 3′ expression level control probe and a 5′ expression level control probe that specifically hybridize to a particular housekeeping gene is used as an indicator of the efficiency of cRNA preparation. A ratio of 1-3 indicates an acceptable preparation.

Databases

Any appropriate computer platform may be used to perform the necessary comparisons between sequence information, gene expression information and any other information in a database or provided as an input. For example, a large number of computer workstations and programs are available from a variety of manufacturers, such has those available from Affymetrix.

Statistical Methods

Combining profiles of gene expression over a wide array of transcripts has potentially more classification prediction power than relying on any single gene. This contention relies implicitly on the intricate nature of gene-to-gene interactions and the host of possible molecular characteristics captured in genome wide RNA expression. The significance of the difference between the levels of gene expression between tissue sample types can be assessed using expression data and any number of statistical tests such as Significance Analysis of Microarrays (SAM) method (Tusher V G, et al., 2001, Proc. Natl. Acad. Sci. USA 98(9):5116-21). SAM identifies genes with statistically significant changes in expression by assimilating a set of gene-specific t-tests. Each gene is assigned a score on the basis of its change in gene expression relative to the standard deviation of repeated measurements for that gene. Genes with scores greater than a threshold are deemed potentially significant. The percentage of such genes identified by chance is the false discovery rate (FDR). To estimate the FDR, nonsense genes are identified by analyzing permutations of the measurements. The threshold can be adjusted to identify smaller or larger sets of genes, and FDRs are calculated for each set.

Kits

Any of the compositions described herein may be comprised in a kit. In a non-limiting example, reagents for determining the genotype of one or more of the fourteen genes listed in Table 3 are included in a kit. The kit may further include individual nucleic acids that can be amplify and/or detect particular nucleic acid sequences of one or more of the fourteen genes listed in Table 3 gene. It may also include one or more buffers, such as a DNA isolation buffers, an amplification buffer or a hybridization buffer. The kit may also contain compounds and reagents to prepare DNA templates and isolate DNA from a sample. The kit may also include various labeling reagents and compounds.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

A kit will also include instructions for employing the kit components as well the use of any other reagent not included in the kit. Instructions may include variations that can be implemented.

It is contemplated that such reagents are embodiments of kits of the invention. Such kits, however, are not limited to the particular items identified above and may include any reagent used directly or indirectly in the detection of all fourteen genes listed in Table 3.

### Example 1

### Patients, Samples and Treatment

Selection of the Patients

Patients were selected according to the following eligibility criteria:

Patients with histologically-proven colorectal cancer;
Patients treated as a fist line treatment with a combination of irinotecan and 5FU according to FOLFIRI schedule;
Available clinical and histopathological data;
Chemotherapeutic response determined according to WHO (or RECIST) criteria or data allowing to evaluate the response must be available; and
Available frozen tumor material or RNA sample

Patients were excluded from the study if they:
were previously treated with a topoisomerase I inhibitor (irinotecan, topotecan)
had previous lines of chemotherapy for treatment of metastases
had no clinical and histopathological data available
had no frozen tumor material or RNA sample available.
had inadequate RNA quality or quantity upon isloation.

Inclusion Procedure

Clinical data and sample (frozen tumor or RNA) collection was performed according to the following guidelines:
Determine the number of colorectal cancers patients for which frozen tumor sample is available
Among said patients, determine the number of colorectal cancer patients with synchronous metastases treated with FOLFIRI as a first line treatment
Retrieve frozen tumor material or RNA sample and transfer samples as soon as possible, on dry ice.
Extract RNA (if necessary) and assay on RNA 6000 Nano LabChips® to get reliable information on RNA quality according to a standardized procedure set up at the laboratory
If enough high quality RNA is obtained, all clinical and histopathological data for the corresponding patient is annotated as indicated on a data collection sheet.
paraffin-embedded material of the tumor is then collected.

The tumor sample validation is an essential step to ensure that the frozen material represents true invasive carcinoma, without adenoma component. Moreover this analysis is crucial for the precise determination of the percentage of tumor cells, of necrosis and fibrosis. Finally this step determines the specificity of the tissue that will be analysed and guaranties the amount of available materiel.

Twenty-nine colorectal cancer patients with synchronous and unresectable liver metastases were treated, as first-line treatment, with a combination of irinotecan, fluorouracil, and leucovorin (FOLFIRI) at CRLC Val d'Aurelle, France. Ten patients were participants in a multicenter prospective phase II clinical trial (high-dose FOLFIRI) aimed at assessing the efficacy and safety of increasing dose of irinotecan (from 180 to 260 mg/m²) combined with the simplified lecovorin (“LV”) and fluorouracil (“5FU”) regimen in first line patients with metastatic colorectal cancer. The remaining patients received a FOLFIRI regimen with a standard dose of irinotecan (180 mg/m²). For one patient, intravenous 5-FU was replaced by an oral form of 5-FU (5-fluorouracil (5-FU) prodrug tegafur with uracil or UFT). Before any chemotherapy, all patients underwent surgery for primary tumor resection. We collected 29 colon tumor samples following a standardized procedure in order to obtain high quality RNA. Five samples were excluded on the basis of poor quality RNA (2), low quantity RNA (1) and poor chip expression quality (2). Also excluded were two samples from a single patient with two different localizations of his primary tumor and one sample from a patient who died during treatment. Thus, only 21 samples were eligible for further transcriptome analysis.

Measurement of the target lesion in the tumor response evaluations was performed in accordance with the World Health Organization (WHO) recommendations for the evaluation of cancer treatment in solid tumors (Miller A B, et al., 1981 Cancer 47(1):207-14). Using computed tomography scanning, metastatic tumor size was estimated from bidimensional measurements (product of longest perpendicular diameters) before and after each 4 or 6 cycles of chemotherapy to calculate the percentage of change from baseline. Patients with a decrease of metastatic tumor size greater than 50% were classified as responders (R), and patients with a decrease of metastatic tumor size less than 50% or with an increase in size of lesions were classified as non-responders (NR). Evaluation of the tumor response of the 21 patients is summarized in Table 1.

TABLE 1
Evaluation of tumor response

| Identification | % of target | Evaluation | |
|---|---|---|---|
| of patients | lesion change | response | Status |
| 130-YL | −94 | PR | R |
| 149-JG-I | −86 | PR | R |
| 016-MV | −84 | PR | R |
| 044-MB | −80 | PR | R |
| 022-JB | −79 | PR | R |
| 061-CM | −77 | PR | R |
| 115-CB | −69 | PR | R |
| 059-MT | −65 | PR | R |
| 244-FP | −52 | PR | R |
| 222-PEM | −44 | SD | NR |
| 119-PM | −39 | SD | NR |
| 223-GB | −29 | SD | NR |
| 196-TD | −27 | SD | NR |
| 73-PD | −20 | SD | NR |
| 189-JR | −19 | SD | NR |
| 94-AM | −15 | SD | NR |
| 056-MC | −14 | SD | NR |
| 213-RG | −4 | SD | NR |
| 045-JC | 0 | SD | NR |
| 227-SS | 0 | SD | NR |
| 89-NC | +25 | PD | NR |

CR = complete response,
PR = partial response (decrease ≧ 50%),
SD = stable disease (neither PR or PD criteria met),
PD = progression disease (increase ≧ 25% or appearance of new lesions);
CR and PR have to be confirmed at 4 weeks
R = responder;
NR = non-responder

### Example 2

### Assessment of Clinical Response

Before doing gene expression analysis, responder and non-responder patients were defined based upon anatomic indicators (tumor lesions) according to WHO criteria. We have considered the best response to first-line chemotherapy. Of these 21 patients, 9 (43%) were sensitive to FOLFIRI treatment showing a size reduction of metastases from 52% to 94% whereas 12 (57%) were considered as non-responders with tumor size decrease no more than 44% or tumor size increase up to 25% (Table 1).

To assess differences in clinicopathological features between responder and non-responder patients we used a Fisher's exact test for qualitative variables and a non-parametric Wilcoxon test for quantitative ones. As shown in Table 2 patient and tumor characteristics did not differ significantly between both groups.

TABLE 2
Clinical and pathological characteristics of patients

| | | | Non- | | | | |
|---|---|---|---|---|---|---|---|
| | Responders | | responders | | Total | | |
| | (N = 9) | | (N = 12) | | (N = 21) | | |
| Characteristics | N | (%) | N | (%) | N | (%) | p |
| Sex | | | | | | | |
| men | 3 | 33.3 | 8 | 66.7 | 11 | 52.4 | 0.198 |
| women | 6 | 66.7 | 4 | 33.3 | 10 | 47.6 | |
| Age, median [min-max] | 57 [45-68] | | 62 [50-71] | | 60 [45-71] | | 0.136 |
| Tumor localisation | | | | | | | |
| Right colon | 1 | 11.1 | 0 | 0 | 1 | 4.8 | 0.83 |
| Transverse colon | 1 | 11.1 | 1 | 8.3 | 2 | 9.5 | |
| Left colon | 7 | 77.8 | 10 | 83.4 | 17 | 81 | |
| Rectum-sigmoid | 0 | 0 | 1 | 8.3 | 1 | 4.7 | |
| junction | | | | | | | |
| Differentiation | | | | | | | |
| Well | 5 | 55.6 | 4 | 33.3 | 9 | 42.9 | 0.910 |
| Moderate | 3 | 33.3 | 5 | 41.7 | 8 | 38.1 | |
| Poor | 1 | 11.1 | 2 | 16.7 | 3 | 14.3 | |
| ND | 0 | 0 | 1 | 8.3 | 1 | 4.7 | |
| pN | | | | | | | |
| N0 | 1 | 11.1 | 3 | 25 | 4 | 19.05 | 0.842 |
| N1 | 2 | 22.2 | 2 | 16.7 | 4 | 19.05 | |
| N2 | 6 | 66.7 | 7 | 58.3 | 13 | 61.9 | |
| pT | | | | | | | |
| T3 | 8 | 88.9 | 8 | 66.7 | 16 | 76.2 | 0.338 |
| T4 | 1 | 11.1 | 4 | 33.3 | 5 | 23.8 | |
| Therapeutic schedule | | | | | | | |
| FOLFIRI | 2 | 22.2 | 8 | 66.7 | 10 | 47.6 | 0.05 |
| High IRI | 7 | 77.8 | 3 | 25 | 10 | 47.6 | |
| UFT-COMPTO | 0 | 0 | 1 | 8.3 | 1 | 4.8 | |
| WHO performance status | | | | | | | |
| 0 | 4 | 44.4 | 5 | 41.7 | 9 | 42.9 | 1 |
| 1 | 5 | 55.6 | 7 | 58.3 | 12 | 57.1 | |
| CEA (pretherapeutic) | 112 | | 92 [1-1129] | | 102 | | 0.518 |
| median [min-max] | [5-36812] | | | | [1-36812] | | |
| ≦10 ng/ml | 1 | 11.1 | 4 | 36.4 | 5 | 25 | 0.319 |
| >10 ng/ml | 8 | 88.9 | 7 | 63.6 | 15 | 75 | |
| LDH (pretherapeutic) | 660 | | 534 | | 563.5 | | 0.711 |
| median [min-max] | [259-3238] | | [276-3992] | | [259- | | |
| | | | | | 3992] | | |
| ≦480 U/L | 3 | 42.9 | 3 | 33.3 | 6 | 37.5 | 1 |
| >480 U/L | 4 | 57.1 | 6 | 66.7 | 10 | 62.5 | |
| Number of metastatic | | | | | | | |
| sites | | | | | | | |
| 1 | 9 | 100 | 9 | 75 | 18 | 85.7 | 0.486 |
| 2 | 0 | 0 | 1 | 8.3 | 1 | 4.8 | |
| 3 | 0 | 0 | 2 | 16.7 | 2 | 9.5 | |

### Example 3

### Assay Methods

All tissue samples were maintained at −180° C. (liquid nitrogen) or at −80° C. until RNA extraction and were weighed before homogenization. Then tissue samples were disrupted directly into a lysis buffer using Mixer Mill® MM 300 (Qiagen, Valencia, Calif.). The denaturing agents present into the lysis buffer inactivate cellular nucleases during cells or tissus disruption while maintaining RNA integrity. Total RNA was isolated from tissue lysates using RNeasy® mini Kit (Qiagen), and additional DNAse digestion was performed on all samples during the extraction process (RNase-Free DNase Set™ Protocol for DNase treatment on RNeasy® Mini spin columns, Qiagen). After each extraction, a small fraction of the total RNA preparation was taken to determine the quality of the sample and the yield of total RNA. Controls were performed by UV spectroscopy and analysis of total RNA profile using Agilent RNA 6000 Nano LabChip® kit with Agilent 2100 Bioanalyser (Agilent Technologies, Palo Alto, Calif.) to determine RNA purity, quantity, and integrity.

### Example 4

### Gene Expression Analysis

Total RNA was labeled according to standard Affymetrix protocols (see Affymetrix GeneChip® Expression Analysis Technical Manual; Affymetrix Inc., Santa Clara, Calif.). Generally, total RNA or mRNA was first reverse transcribed using a T7-Oligo(dT) Promoter Primer in the first-strand cDNA synthesis reaction. Following RNase H-mediated second-strand cDNA synthesis, the double-stranded cDNA was purified and serves as a template in the subsequent in vitro transcription (IVT) reaction. The IVT reaction was carried out in the presence of T7 RNA Polymerase and a biotinylated nucleotide analog/ribonucleotide mix, for complementary RNA (cRNA) amplification and biotin labeling. The biotinylated cRNA targets were then cleaned up, fragmented, and hybridized to GeneChip® expression arrays. For each sample, the labeled probes were then hybridized onto the Affymetrix Human Genome U133 Set (HG-U133; Affymetrix Inc., Santa Clara, Calif.), which contains 44,298 probe sets representing more than 39,000 transcripts derived from approximately 33,000 well-substantiated human genes. Hybridization and was performed using an Affymetrix GeneChip® Station and the conditions were as recommended in the Affymetrix GeneChip® Expression Analysis Technical Manual. After hybridization, the chips were stained with streptavidin phycoerythrin conjugate and scanned by the GeneChip® Scanner 3000 or the GeneArray® Scanner, where the amount of light emitted at 570 nm is proportional to the bound target at each location on the probe array. Inter-array normalization was performed using a set of standard genes with low variability common to the arrays, provided by Affymetrix, and applying a scaling factor for each array. The final data set file was complied using Affymetrix GeneChip® software, which, for each probe set, assigned an intensity corresponding to transcript abundance.

Expression profiling was conducted using Affymetrix U133 A and B chips comprised of 44298 probes set. For statistical analysis genes present in at least 50% of patients from one group were considered for further analysis resulting in a list of 19365 genes.

### Example 5

### Determination of Gene Signature

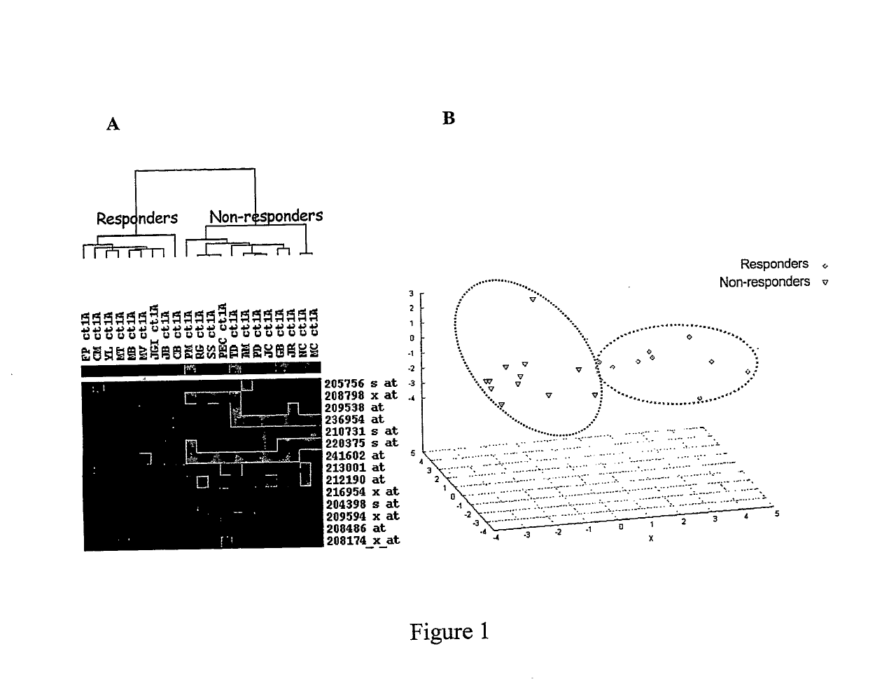

The differentially expressed genes between responders and non-responders were determined using SAM. Based on a relevant FDR of 20%, about 5000 discriminatory genes were selected and ranked according their statistical significance. For each gene, using a non-parametric procedure, the total area (AUG) was estimated and the partial area (pAUC) under the receiver operating characteristic (ROC) curve was determined. The estimation of the pAUC has been restricted only to the region where the specificity is at least 90%. Genes were then ranked according to AUC and pAUC values and for each indicator we retained the top 40 genes. This process was repeated twenty one times with a training set of 20 samples (each time, a sample was held out). In order to establish a stable signature we selected the genes common to the 21 AUC lists (8 genes) and those common to the 21 pAUC lists (11 genes). Finally, as some genes were common to both the final AUC and pAUC lists, a set of 14 discriminatory genes were retained (Table 3). Unsupervised hierarchical clustering and Principal Component Analysis were applied to the 14 selected genes and this resulted, in both analyses, in a clear separation between responder and non-responders patients (FIG. 1).

TABLE 3
The 14-gene signature that predicts response to FOLFIRI

| | | | GO Molecular | | | Fold |
|---|---|---|---|---|---|---|
| Probe set | Gene | | Function | | | change |
| ID | Symbol | Gene description | Description | pAUC | AUC | R/NR |
| 210731_s_at | LGALS8 | Consensus includes | sugar binding/ | 0.083* | 0.907 | 1.83 |
| | | gb: AL136105/DEF = Human | sugar binding | | | |
| | | DNA sequence from clone RP4- | | | | |
| | | 670F13 on chromosome | | | | |
| | | 1q42.2-43. Contains the | | | | |
| | | gene for Po66 carbohydrate | | | | |
| | | binding protein similar to | | | | |
| | | soluble galactoside-binding | | | | |
| | | lectin 8 (galectin 8, LGALS8), | | | | |
| 212190_at | SERPINE2 | Consensus includes | serine-type | 0.075 | 0.935** | 2.31 |
| | | gb: AL541302/FEA = EST/ | endopeptidase | | | |
| | | DB_XREF = gi: 12872241/ | inhibitor activity/ | | | |
| | | DB_XREF = est: AL541302/ | heparin binding | | | |
| | | CLONE = CS0DE006YI10 | | | | |
| 213001_at | ANGPTL2 | Consensus includes | receptor binding | 0.092* | 0.972** | 1.94 |
| | | gb: AF007150.1/ | | | | |
| | | DEF = Homo sapiens clone | | | | |
| | | 23767 and 23782 mRNA | | | | |
| | | sequences. | | | | |
| 216954_x_at | ATP5O | Consensus includes | transporter | 0.075 | 0.944** | 1.61 |
| | | gb: S77356.1/DEF = | activity/hydrolase | | | |
| | | Homo sapiens oligomycin | activity/hydrogen- | | | |
| | | sensitivity conferral | transporting ATP | | | |
| | | protein oscp-like protein | synthase activity | | | |
| | | mRNA, partial cds. | | | | |
| 220375_s_at | PRYM | gb: NM_024752.1/ | | 0.092* | 0.981** | 2.07 |
| | | DEF = Homo sapiens | | | | |
| | | hypothetical protein | | | | |
| | | FLJ23312 (FLJ23312), | | | | |
| | | mRNA. | | | | |
| 204398_s_at | EML2 | gb: NM_012155.1/ | — | 0.083* | 0.88 | 1.49 |
| | | DEF = Homo sapiens | | | | |
| | | microtubule-associated | | | | |
| | | protein like echinoderm | | | | |
| | | EMAP (EMAP-2), mRNA. | | | | |
| 205756_s_at | F8 | gb: NM_000132.2/ | copper ion | 0.083* | 0.917 | 1.82 |
| | | DEF = Homo sapiens | binding/ | | | |
| | | coagulation factor VIII, | oxidoreductase | | | |
| | | procoagulant component | activity | | | |
| | | (hemophilia A) (F8), | | | | |
| | | transcript variant 1, mRNA. | | | | |
| 208174_x_at | U2AF1L2 | gb: NM_005089.1/ | nucleotide | 0.092* | 0.944** | 1.32 |
| | | DEF = Homo sapiens U2 | binding/RNA | | | |
| | | small nuclear | binding | | | |
| | | ribonucleoprotein auxiliary | | | | |
| | | factor, small subunit 2 | | | | |
| | | (U2AF1RS2), mRNA. | | | | |
| 208486_at | DRD5 | gb: NM_000798.1/ | rhodopsin-like | 0.083* | 0.889 | 1.33 |
| | | DEF = Homo sapiens | receptor activity/ | | | |
| | | dopamine receptor D5 | receptor activity/ | | | |
| | | (DRD5), mRNA. | dopamine receptor | | | |
| | | | activity | | | |
| 208798_x_at | GOLGIN-67 | gb: AF204231.1/ | — | 0.083* | 0.926 | 1.67 |
| | | DEF = Homo sapiens 88- | | | | |
| | | kDa Golgi protein (GM88) | | | | |
| | | mRNA, complete cds. | | | | |
| 209538_at | ZNF32 | gb: U69645.1/DEF = Human | nucleic acid | 0.083* | 0.972** | 2.09 |
| | | zinc finger protein mRNA, | binding/DNA | | | |
| | | complete cds. | binding/zinc | | | |
| | | | ion binding | | | |
| 209594_x_at | PSG9 | gb: M34421.1/DEF = Human | — | 0.083* | 0.87 | 1.62 |
| | | pregnancy-specific beta-1 | | | | |
| | | glycoprotein mRNA, | | | | |
| | | complete cds. | | | | |
| 236954_at | BOLL | Consensus includes | nucleotide | 0.075 | 0.972** | 70.75 |
| | | gb: BF059752/FEA = EST/ | binding/nucleic | | | |
| | | DB_XREF = gi: 10813648/ | acid binding/ | | | |
| | | DB_XREF = est: 7k65h06.x1/ | RNA binding | | | |
| | | CLONE = IMAGE: 3480442/ | | | | |
| | | UG = Hs.169797 ESTs | | | | |
| 241602_at | ZNF582 | Consensus includes | nucleic acid | 0.083* | 0.935** | 161.31 |
| | | gb: BG432829/FEA = EST/ | binding/zinc | | | |
| | | DB_XREF = gi: 13339335/ | ion binding | | | |
| | | DB_XREF = est: 602496037 | | | | |
| | | F1/CLONE = IMAGE: 4610000/ | | | | |
| | | UG = Hs.152174 ESTs | | | | |

*Genes selected by pAUC;
**Genes selected by AU

Using an SVM-learning algorithm, a predictor classifier was defined and its performance was evaluated by the “LOOCV”. All the 9 responders (100% specificity) and 11 out of 12 non-responders (92% sensitivity) were correctly classified, for an overall accuracy of 95% to response to treatment.

PREDICTIVE VALUES

| | | | Gold Standard |
|---|---|---|---|
| | | | (WHO criteria) |
| | | NR | R |
| Prediction | positive: NR | TP = 11 | FP = 0 |
| (Signature) | negative: R | FN = 1 | TN = 9 |
| | | 12 | 9 |

TP = true positives;
FP = false positives;
FN = false negatives;
TN = true negatives

Sensitivity is defined as TP/(TP+FN); which is referred to as the “true positive rate”. The sensitivity (Se) corresponds to the proportion to the proportion of positive results among the NR patients.

$$Se = \frac{11}{12} = 0.92$$

Specificity is defined as TN/(TN+FP); which is referred to as the “true negative rate”. The specificity (Sp) corresponds to the proportion of negative results among the R patients.

$$Sp = \frac{9}{9} = 1$$

The positive predictive value (PPV) of a diagnostic test corresponds to the probability of a NR status if the signature gives a positive result. It is calculated by:

$$PPV = \frac{Se \times pvr}{Se \times pvr + ( 1 - Sp ) ( 1 - pvr )}$$

where, “prv” corresponds to the prevalence of NR status, estimated by the proportion of NR patients in the population. In this example,

$$pvr = \frac{12}{21} = 0.57 \% . \\ PPV = \frac{Se \times \frac{12}{21}}{Se \times \frac{12}{21} + ( 1 - Sp ) ( 1 - \frac{12}{21} )} = 1$$

$$PPV = 100 \% .$$

The negative predictive value (NPV) of a diagnostic test corresponds to the probability of a R status if the signature gives a negative result. It is calculated by

$$\begin{matrix}NPV = 1 - \frac{( 1 - Se ) \times \frac{12}{21}}{( 1 - Se ) \times \frac{12}{21} + Sp \times ( 1 - \frac{12}{21} )} \\ = 1 - \frac{\frac{1}{21}}{\frac{1}{21} + \frac{9}{21}} \\ = 1 - \frac{1}{10} \\ = \frac{9}{10} \\ = 0.9\end{matrix}$$

$$NPV = 90 \%$$

To assess the misclassification rates, the approach described by Michiels 31 is utilized in accordance with Michiels S, Koscielny S, Hill C: Prediction of cancer outcome with microarrays: a multiple random validation strategy. Lancet 365:488-492, 2005, incorporated herein by reference.

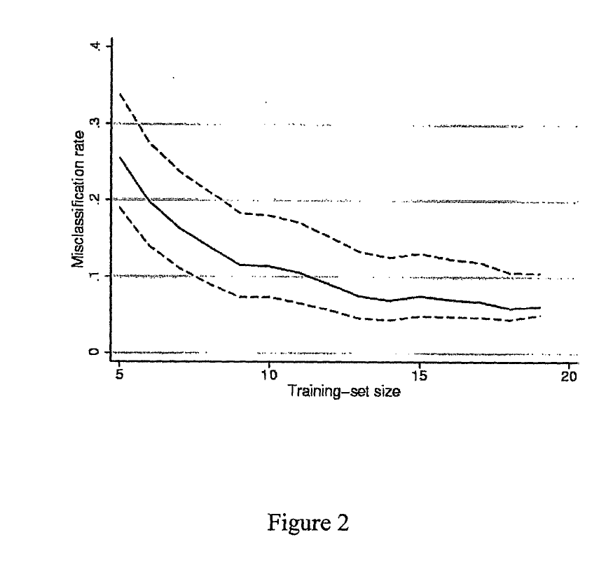

This method permits the determination of a mean rate of misclassification and plots this proportion of misclassification in validation sets as a function of the corresponding training set size (see FIG. 2)

This method consists in dividing the dataset into training sets of different size (from 5 to 19 samples) with at least one patient of each outcome. The remaining samples were considered as validation set (size from 16 to 2). 500 random training set were associated with each sample size. For a given training set, a classifier was built by SVM using the 14 selected genes and tested in a designated validation test. As shown in FIG. 2, even with the smallest training size, the misclassification rate was only 25.6% (95 Cl 19%-33.8%) and from a training set size >13, the misclassification rate did not exceed 7.5%.

First, we only considered genes called present in at least 50% of the patients from any one group. Data analysis was performed on the 19,365 remaining genes to determine an expression profile able to predict responder's patients. Differentially expressed genes between responders and non-responders were detected by means of the “Significance Analysis of Microarrays” method (SAM28). This approach allowed calculation of a d-score which corresponds to a Student's statistic with a factor added to the classic denominator. Then, genes were classified genes according to this score and their statistical significance. A set of genes with a relevant “False Discovery Rate” (FDR) of 20% were also identified.

The selected genes as a result of the SAM method was then ranked by computing the empirical area under the Receiver Operating Characteristic (ROC) curve (AUC) and the empirical partial AUC (pAUC) restricted to a clinically relevant pertinent range of false-positive rates 29. The pAUC is an index of discrimination and the interval of chosen false positive rates allows considering a high specificity in order to particularly well detect the responder population. Then, the classification rule was defined with Support Vector Machines algorithm 30. Two parameters were required, the kernel function (RBF) and the magnitude of the penalty for violating the soft margin. Finally, leave-one-out cross validation (LOOCV) was used to estimate the performance and the accuracy of the output class prediction rule. With LOOCV, one sample is left out, and the remaining samples were used to construct a predictor classifier, which is used to classify the left-out sample.

### Example 6

Functional classification of 14 genes from signature All the 14 genes from signature were over-expressed in responder tumors. These genes showed a wide ratio as 1.3-160-fold increases in expression in sensitive compared with resistant tumors. According to GeneOntology classification, functional classes of these differentially expressed genes included RNA splicing (U2AF1L2), regulation of transcription (ZNF32 and ZNF582), cell adhesion (F8, Galectin-8, PSG9), cell differentiation (SERPINE2, BOLL), ion transport (ATP5O), signal transduction (DRD5) development (ANGPTL2) and visual perception (EML2). GOLGIN-67 is a membrane Golgi protein whose function is unknown.

Among the 14 genes, three genes, galectine-8, PSG9 and SERPINE2 (or PN-1), could be involved in the adhesion process. Galectin-8 is a matricellular protein that positively or negatively regulates cell adhesion, depending on the extracellular context 35. Moreover, the quantitative determination of the immunohistochemical expression of galectin-8 in the series of colon cancer specimens clearly showed that the extensively invasive colon cancers exhibited significantly less galectin-8 than locally invasive ones 36. PSG9, which is ectopically upregulated in vivo by colon cancer cells, 37 has an RGD motif in a conserved region in the N-terminal domain which suggests that these genes may function as adhesion recognition signals for integrins and are involved in adhesion/recognition processes 38. The serine proteinase inhibitor SERPINE2 could participate in maintaining the integrity of connective tissue matrices. SERPINE2 has been shown to inhibit tumour cell-mediated extracellular matrix destruction 39. Two other genes, FVIII and ANGPTL2, could reflect the tumour vascularization. Indeed, intratumoral angiogenesis is commonly quantified by microvessel density measurement using immunohistochemical staining with monoclonal antibodies against factor VIII 40. ANGPTL2 protein induces sprouting in vascular endothelial cells 41 and promotes angiogenesis 42. Altogether, these results support the idea that the responders tumour seems more adhesive and vascularized than the non-responder's one.

1. A method of predicting response of a human patient with colorectal cancer to chemotherapy, comprising detecting the expression of one or more genes selected from the group consisting of LGALS8, SERPINE2, ANGPTL2, ATP50, PRYM, EML2, F8, U2AF1L2, DRD5, GOLGIN-67, ZNF32, PSG9, BOLL, and ZNF583 in a tumor tissue sample from the patient wherein said gene expression is indicative of said patient's response to chemotherapy.

2. The method of claim 1, comprising detecting the expression of 2 or more genes.

3. The method of claim 1, comprising detecting the expression of 3 or more genes.

4. The method of claim 1, comprising detecting the expression of 4 or more genes.

5. The method of claim 1, comprising detecting the expression of 5 or more genes.

6. The method of claim 1, comprising detecting the expression of 6 or more genes.

7. The method of claim 1, comprising detecting the expression of 7 or more genes.

8. The method of claim 1, comprising detecting the expression of 8 or more genes.

9. The method of claim 1, comprising detecting the expression of 9 or more genes.

10. The method of claim 1, comprising detecting the expression of 10 or more genes.

11. The method of claim 1, comprising detecting the expression of 11 or more genes.

12. The method of claim 1, comprising detecting the expression of 12 or more genes.

13. The method of claim 1, comprising detecting the expression of 13 or more genes.

14. The method of claim 1, wherein the gene is selected from the group consisting of SERPINE2, ANGPTL2, ATP50, PRYM, EML2, F8, U2AF1L2, DRD5, GOLGIN-67, ZNF32, PSG9, BOLL, and ZNF583.

15. The method of claim 14, wherein the gene is selected from the group consisting of ANGPTL2, ATP50, EML2, F8, U2AF1L2, DRD5, GOLGIN-67, ZNF32, PSG9, BOLL, and ZNF583.

16. The method of claim 15, wherein the gene is selected from the group consisting of ATP50, EML2, F8, U2AF1L2, DRD5, GOLGIN-67, ZNF32, PSG9, BOLL, and ZNF583.

17. The method of claim 16, wherein the gene is selected from the group consisting of PRYM, EML2, F8, U2AF1L2, DRD5, GOLGIN-67, ZNF32, PSG9, BOLL, and ZNF583.

18. The method of claim 17, wherein the gene is selected from the group consisting of EML2, F8, U2AF1L2, DRD5, GOLGIN-67, ZNF32, PSG9, BOLL, and ZNF583.

19. The method of claim 18, wherein the gene is selected from the group consisting of F8, U2AF1L2, DRD5, GOLGIN-67, ZNF32, PSG9, BOLL, and ZNF583.

20. The method of claim 19, wherein the gene is selected from the group consisting of U2AF1L2, DRD5, GOLGIN-67, ZNF32, PSG9, BOLL, and ZNF583.

21. The method of claim 20, wherein the gene is selected from the group consisting of U2AF1L2, DRD5, GOLGIN-67, ZNF32, PSG9, BOLL, and ZNF583.

22. The method of claim 21, wherein the gene is selected from the group consisting of DRD5, GOLGIN-67, ZNF32, PSG9, BOLL, and ZNF583.

23. The method of claim 22, wherein the gene is selected from the group consisting of GOLGIN-67, ZNF32, PSG9, BOLL, and ZNF583.

24. The method of claim 23, wherein the gene is selected from the group consisting of ZNF32, PSG9, BOLL, and ZNF583.

25. The method of claim 24, wherein the gene is selected from the group consisting of PSG9, BOLL, and ZNF583.

26. The method of claim 25, wherein the gene is selected from the group consisting of BOLL, and ZNF583.

27. The method of claim 1, wherein said chemotherapy comprises administering a regimen of irinotecan, fluorouracil, and leucovorin to the patient.

28. The method of claim 1, wherein said chemotherapy comprises administering a pharmaceutical regimen of oxaliplatin, fluorouracil, and leucovorin to the patient.

29. A method of claim 1 wherein detecting the expression of any one or more of the genes LGALS8, SERPINE2, ANGPTL2, ATP50, PRYM, EML2, F8, U2AF1L2, DRD5, GOLGIN-67, ZNF32, PSG9, BOLL, and ZNF583 comprises detecting a protein derived from said genes in a tumor tissue sample from the patient wherein said gene expression is indicative of said patient's response to chemotherapy.

30. A method of determining a chemotherapy regime for a human patient with colorectal cancer, comprising:
a) detecting the expression of one or more genes selected from the group consisting of LGALS8, SERPINE2, ANGPTL2, ATP50, PRYM, EML2, F8, U2AF1L2, DRD5, GOLGIN-67, ZNF32, PSG9, BOLL, and ZNF583 from a tumoral tissue sample from the patient wherein said gene expression is predicative of response to chemotherapy; and
b) administering a regimen comprising irinotecan, fluorouracil, and leucovorin to said patient if one or more of the genes listed in step (a) is detected in said patient.

31. A method of determining a chemotherapy regime for a human patient with colorectal cancer, comprising:
a) detecting the expression of one or more genes selected from the consisting of group LGALS8, SERPINE2, ANGPTL2, ATP50, PRYM, EML2, F8, U2AF1L2, DRD5, GOLGIN-67, ZNF32, PSG9, BOLL, and ZNF583 from a tissue sample from the patient wherein said gene expression is indicative of response to chemotherapy; and
b) administering a regimen comprising oxaliplatin, fluorouracil, and leucovorin to said patient if one or more of the genes listed in step (a) is not detected in said patient.

32. A method of modifying a chemotherapy treatment for a human patient with colorectal cancer, comprising:
a) administering a chemotherapy regimen to the patient;
b) detecting the expression of one or more genes selected from the following group consisting of LGALS8, SERPINE2, ANGPTL2, ATP50, PRYM, EML2, F8, U2AF1L2, DRD5, GOLGIN-67, ZNF32, PSG9, BOLL, and ZNF583 from a tissue sample from the patient; and
c) administering irinotecan, fluorouracil, and leucovorin to said patient when one or more genes identified in (b) are expressed or administering oxaliplatin, fluorouracil, and leucovorin to said patient when one or more genes identified in (b) are not expressed.

33. A method of claim 1 wherein said method comprises detecting a response of said human patient with metastatic colorectal cancer to chemotherapy.

34. A method of claim 30 wherein said pharmaceutical regime comprises administering to said patient irinotecan, fluorouracil, and leucovorin.

35. A method of claim 30 wherein said pharmaceutical regime comprises administering to said patient oxaliplatin, fluorouracil, and leucovorin.

36. A kit for use to select the optimal chemotherapy from several alternative treatment options for a human patient with colorectal cancer, the kit comprising:
a. a microarray for detecting a mRNA derived from a sample from said human to assess the expression of the one or more of a gene selected from the group consisting of LGALS8, SERPINE2, ANGPTL2, ATP50, PRYM, EML2, F8, U2AF1L2, DRD5, GOLGIN-67, ZNF32, PSG9, BOLL, and ZNF583; and
b. instructions describing a method of using said microarray.

37. A kit as in claim 36 wherein the microarray is a gene chip.

38. A kit for use to select the optimal chemotherapy from several alternative treatment options for a human patient with colorectal cancer, the kit comprising:
a. a Western blot kit for detecting a protein derived from a sample from said human to assess the expression of the one or more of a gene selected from the group consisting of LGALS8, SERPINE2, ANGPTL2, ATP50, PRYM, EML2, F8, U2AF1L2, DRD5, GOLGIN-67, ZNF32, PSG9, BOLL, and ZNF583; and
b. instructions describing a method of using said Western blot kit.

39. A kit of claim 36 for use to capable of determining the optimal chemotherapy from several alternative treatment options for a human patient with metastatic colorectal cancer.
